(19) United States
(12) Patent Application Publication
(10) Pub. No.: US 2023/0210542 A1
(43) Pub. Date: Jul. 6, 2023

(54) Methods and Systems for Treating Femoroacetabular Impingement

(71) Applicants: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Singapore (SG)

(72) Inventors: Nathan Anil NETRAVALI, Littleton, MA (US); Brian William QUIST, Salem, NH (US); Nicholas Ryan LABRIOLA, ANDOVER, MA (US); Allison Marie STAUFFER, BRIGHTON, MA (US)

(73) Assignees: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Singapore (SG)

(21) Appl. No.: 18/001,421

(22) PCT Filed: Jun. 29, 2021
(86) PCT No.: PCT/US2021/039505

§ 371 Date: Dec. 9, 2022

Related U.S. Application Data
(60) Provisional application No. 63/047,319, filed on Jul. 2, 2020.

(51) Int. Cl. A61B 17/16; A61B 34/10; A61B 34/20

(52) U.S. Cl. CPC A61B 17/16; A61B 17/1666; A61B 17/1668; A61B 34/10; A61B 34/20; A61B 2034/104; A61B 2034/105; A61B 2034/2055; A61B 2017/00123

(57) ABSTRACT

Treating femoroacetrabular impingement. At least one example is a method comprising: monitoring, by a procedure controller, location of a first member of an acetabulofemoral joint in a three-dimensional coordinate space; tracking, by the procedure controller, an amount of bone resected from the first member of the acetabulofemoral joint by tracking a distal end of a resection device in the three-dimensional coordinate space; and controlling, by the procedure controller, a rate of resection of the resection device based on the location of the distal end of the resection device relative to a planned resection volume associated the first member of the acetabulofemoral joint.

### CROSS-REFERENCE TO RELATED APPLICATIONS

This application claims the benefit of U.S. provisional application Ser. No. 63/047,319 filed Jul. 2, 2020 and titled, “Planning and Robotic Assistance for Treatment of Femoroacetabular Impingement.” The provisional application is incorporated by reference herein as if reproduced in full below.

### BACKGROUND

Femoroacetabular impingement (FAI) is a cause of damage to the labrum or articular cartilage of the hip. FAI results from a bony overgrowth on the neck of the femur (called a cam deformity), a bony overgrowth around the acetabular rim (called a pincer deformity), or a combination of the two. Treatment of FAI involves using a mechanical resection device to remove bone and create an anatomical profile that does not result in impingement during typical ranges of motion. Treatment can be performed with respect to the cam deformity, the pincer deformity, or both.

One of the challenges in treating FAI is that it is difficult to determine the appropriate locations and amounts of bone to be removed to reduce the impingement. Multiple X-rays from various angles can characterize the overgrowth around the joint from particular perspectives, but it is difficult to characterize the three-dimensional (3D) nature of the anatomy using only two-dimensional (2D) X-ray images. Given this, one technique is to obtain magnetic-resonance imaging (MRI) or computed tomography (CT) images to view the anatomy in a 3D perspective. Although CT's can be used to construct 3D bone models, allowing the surgeon to see the cam and pincer deformities in their entirety, the 3D bone models do not provide the surgeon with information on how much bone needs to be removed to relieve the impingement. Furthermore, during arthroscopic treatment, it is difficult to determine how much bone has been removed around the circumference of the femoral head and neck through the arthroscopic video. Thus, surgeons may rely heavily on intraoperative fluoroscopy to provide 2D images to determine the silhouette of the bone. By taking these fluoroscopy images in various orientations, an attempt is made to determine if the impingement has resolved.

Because over-resection may lead to femoral neck fracture and/or fracture of the acetabulum, under-resection is common. In fact, considering repeat hip arthroscopy procedures, under-resection is the cause in about 64% of the cases.

### SUMMARY

Treating femoroacetabular impingement. One example is a method of treating femoroacetabular impingement, the method comprising: monitoring, by a procedure controller, location of a first member of an acetabulofemoral joint in a three-dimensional coordinate space; tracking, by the procedure controller, an amount of bone resected from the first member of the acetabulofemoral joint by tracking a distal end of a resection device in the three-dimensional coordinate space; and controlling, by the procedure controller, a rate of resection of the resection device based on the location of the distal end of the resection device relative to a planned resection volume associated with the first member of the acetabulofemoral joint.

In the example method, the first member of the acetabulofemoral joint may be at least one selected from a group comprising: a femur; and an acetabulum.

In the example method, controlling the rate of resection may further comprise decreasing a rotational speed of a cutter of the resection device when the distal end of the resection device resides on a portion of the planned resection volume having less bone to be removed.

In the example method, controlling the rate of resection may further comprise increasing a rotational speed of a cutter of the resection device when the distal end of the resection device resides on a portion of the planned resection volume designating more bone to be removed.

In the example method, controlling the rate of resection may further comprise controlling a rotational speed of a cutter of the resection device based on location of the distal end of the resection device in relation to remaining bone to be removed in the planned resection volume. Controlling the rate of resection may further comprise changing a rotational speed of the cutter of the resection device to zero responsive to the distal end of the resection device abutting bone outside the planned resection volume. Controlling the rate of resection may further comprise changing a rotational speed of the cutter of the resection device to zero responsive to the distal end of the resection device abutting bone in an area beneath the planned resection volume.

The example method may further comprise: creating a three-dimensional model of at least a portion of the first member of the acetabulofemoral joint based on a plurality of images; creating the planned resection volume based on the three-dimensional model; and then providing the three-dimensional model and the planned resection volume to the procedure controller. The plurality of images may be selected from a group comprising: X-ray images; computed tomography images; ultrasound images; and magnetic resonance imaging images. In some cases, prior to monitoring the first member of the acetabulofemoral joint, the example method may comprise registering the first member of the acetabulofemoral joint to correlate the first member to the model.

A second example method of treating femoroacetabular impingement comprises: monitoring, by a procedure controller, location of a first member of an acetabulofemoral joint in a three-dimensional coordinate space; tracking, by the procedure controller, an amount of bone resected by tracking a distal end of a resection device in the three-dimensional coordinate space; and generating, by the procedure controller, a simulated fluoroscopic image that shows the first member of the acetabulofemoral joint after the amount of bone has been removed; and displaying, on a display device, the simulated fluoroscopic image.

The second example method may further comprise: creating a three-dimensional model of at least a portion of the first member of the acetabulofemoral joint based on a plurality of images, the creating the three-dimensional model prior to resecting bone; and providing the three-dimensional model to the procedure controller. The generating the simulated fluoroscopic image may further comprise creating the simulated fluoroscopic image based on the three-dimensional model and the amount of bone resected.

In the example second method, the plurality of images may be selected from a group comprising: x-ray images; computed tomography images; and magnetic resonance imaging images.

In the example second method, generating the simulated fluoroscopic image may further comprise generating a plurality of simulated fluoroscopic images, each image at a different angle relative to the acetabulofemoral joint.

The example second method may further comprise controlling a rate of resection of the resection device based on the location of the distal end of the resection device relative to a planned resection volume associated with the first member of the acetabulofemoral joint. Controlling the rate of resection may further comprise decreasing a rotational speed of a cutter of the resection device when the distal end of the resection device resides on a portion of the planned resection volume having less than a predetermined amount of bone to be removed. Controlling the rate of resection may further comprise increasing a rotational speed of a cutter of the resection device when the distal end of the resection device resides on a portion of the planned resection volume having more than a predetermined amount of bone to be removed. Controlling the rate of resection may further comprise controlling a rotational speed of a cutter of the resection device based on location of the distal end of the resection device in relation to remaining bone to be removed in the planned resection volume. Controlling the rate of resection may further comprise changing a rotational speed of the cutter of the resection device to zero responsive to the distal end of the resection device abutting bone outside the planned resection volume. Controlling the rate of resection may further comprises changing a rotational speed of the cutter of the resection device to zero responsive to the distal end of the resection device abutting bone in an area beneath the planned resection volume.

Another example is a system for treating femoroacetabular impingement, the system comprising: a procedure controller; a stereoscopic camera coupled to the procedure controller; a display device coupled to the procedure controller; a resection controller communicatively coupled to the procedure controller; a resection device operatively coupled to the resection controller, the resection device comprising a handpiece, an elongate outer tube coupled to and extending from the handpiece, and a cutter disposed on a distal end of the elongate outer tube; an optical tracking array coupled to the resection device and in optical view of the stereoscopic camera. The procedure controller may be configured to: monitor location of a first member of an acetabulofemoral joint in a three-dimensional coordinate space; track an amount of bone resected from the first member of the acetabulofemoral joint by tracking a distal end of the resection device in the three-dimensional coordinate space; and control a rate of resection of the resection device based on a location of the distal end of the resection device relative to a planned resection volume associated the first member of the acetabulofemoral joint.

In the example system, when the procedure controller monitors the location of the first member of the acetabulofemoral joint, the procedure controller may be further configured to monitor at least one selected from a group comprising: a femur; and an acetabulum.

In the example system, when the procedure controller controls the rate of resection, the procedure controller may be further configured to decrease a rotational speed of the cutter of the resection device when the distal end of the resection device resides on a portion of the planned resection volume having less than a predetermined amount of bone to be removed.

In the example system, when the procedure controller controls the rate of resection, the procedure controller may be further configured to increase a rotational speed of a cutter of the resection device when the distal end of the resection device resides on a portion of the planned resection volume have more than a predetermined amount of bone to be removed.

In the example system, when the procedure controller controls the rate of resection, the procedure controller may be further configured to control a rotational speed of the cutter of the resection device based on location of the distal end of the resection device in relation to remaining bone to be removed in the planned resection volume. The procedure controller may be further configured to change the rotational speed of the cutter of the resection device to zero responsive to the distal end of the resection device abutting bone outside the planned resection volume. The procedure controller may be further configured to change the rotational speed of the cutter of the resection device to zero responsive to the distal end of the resection device abutting bone in an area beneath the planned resection volume.

In the example system, the procedure controller may be further configured to, prior to controlling the rate of resection, receive a three-dimensional model of at least a portion of the first member of the acetabulofemoral joint in three-dimensions based on a plurality of images; and receive a planned resection volume based on the three-dimensional model.

A second example system may comprise: a procedure controller; a stereoscopic camera coupled to the procedure controller; a display device coupled to the procedure controller; a resection controller communicatively coupled to the procedure controller; a resection device operatively coupled to the resection controller, the resection device comprising a handpiece, an elongate outer tube coupled to and extending from the handpiece, and a cutter disposed on a distal end of the elongate outer tube; and an optical tracking array coupled to the resection device and in optical view of the stereoscopic camera. The procedure controller may be configured to: monitor location of a first member of an acetabulofemoral joint in a three-dimensional coordinate space; track an amount of bone resected by tracking a distal end of the resection device in the three-dimensional coordinate space; generate a simulated fluoroscopic image that shows the first member of the acetabulofemoral joint without the amount of bone resected; and display, on the display device, the simulated fluoroscopic image.

In the second example system, when the procedure controller generates the simulated fluoroscopic image, the procedure controller may be further configured to generate a plurality of simulated fluoroscopic images, each image at a different angle relative to the acetabulofemoral joint.

In the example system, the procedure controller may be further configured to control a rate of resection of the resection device based on a location of the distal end of the resection device relative to a planned resection volume associated with the first member of the acetabulofemoral joint. The procedure controller may be further configured to decrease a rotational speed of the cutter of the resection device when the distal end of the resection device resides on a portion of the planned resection volume having less than a predetermined amount of bone to be removed. The procedure controller may be further configured to increase a rotational speed of the cutter of the resection device as the distal end of the resection device resides on a portion of the planned resection volume having more than a predetermined amount of bone to be removed. The procedure controller may be further configured to control a rotational speed of the cutter of the resection device based on location of the distal end of the resection device in relation to remaining bone to be removed in the planned resection volume. The procedure controller may be further configured to change a rotational speed of the cutter of the resection device to zero responsive to the distal end of the resection device abutting bone outside the planned resection volume. The procedure controller may be further configured to change a rotational speed of the cutter of the resection device to zero responsive to the distal end of the resection device abutting bone in an area beneath the planned resection volume.

### BRIEF DESCRIPTION OF THE DRAWINGS

For a detailed description of example embodiments, reference will now be made to the accompanying drawings in which:

FIG. 1 shows three views of an example femoroacetabular joint;

FIG. 2 shows a system in accordance with at least some embodiments;

FIG. 3 shows an example user interface, in accordance with at least some embodiments;

FIGS. 4A, 4B, 4C, and 4D show a distal end of a resection device in relation to a planned resection volume in accordance with at least some embodiments;

FIG. 5 shows an example user interface, in accordance with at least some embodiments;

FIG. 6 shows a partial block diagram, and partial flow diagram, of a system for treating femoroacetabular impingement, in accordance with at least some embodiments;

FIG. 7 shows a method in accordance with at least some embodiments;

FIG. 8 shows a method of controlling rate of resection of the resection device in accordance with at least some embodiments; and

FIG. 9 shows a computer system in accordance with at least some embodiments.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Various examples are directed to methods and systems for treating femoroacetabular impingement. In particular, various examples are directed to tracking an amount of bone resected from a member of the acetabulofemoral joint by tracking a distal end of the resection device in a three-dimensional coordinate space at the location of the acetabulofemoral joint, and controlling a rate of resection of a resection device based on the location of the distal end of the resection device relative to a planned resection volume associated with the member of the acetabulofemoral joint. The member of the acetabulofemoral joint may be the femur, the acetabulum, or both. For readability the acetabulofemoral joint is hereafter referred as just the “hip joint.” In other examples, based on the tracking of the amount of bone resected from the member of the hip joint, example methods and systems generate a simulated fluoroscopic image that shows the member of the hip joint as the hip joint would look in an actual fluoroscopic image taking into account the resection at any intermediate stage of the intraoperative procedure, to aid the surgeon in determining whether sufficient bone has been removed to address the femoroacetabular impingement. The description first turns to a description of femoroacetabular impingement to orient the reader.

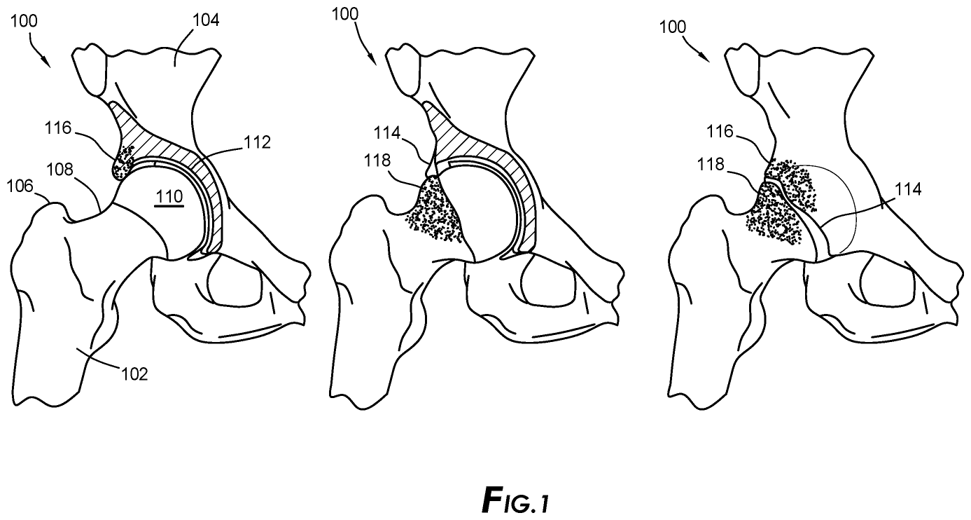

FIG. 1 shows three views of an example hip joint. In particular, visible in each view of FIG. 1 is an example hip joint 100 comprising a portion of a femur 102 and a portion of the acetabulum 104. The portion of the femur 102 that is visible includes the greater trochanter 106 at the upper end of the femur 102, the femoral neck 108 extending from the femur 102, and the femoral head 110 on the distal end of the femoral neck 108. The femoral head 110 is ball-shaped and thus forms the ball of the ball-and-socket joint of the hip joint. The acetabulum 104 defines a spherical-shaped internal surface that forms a socket 112, shown in partial cut-away in the left and middle views of FIG. 1. The femoral head 110 rotates within the socket 112, and the outer edge of the socket 112 is defined by an acetabular rim 114 (middle view).

Femoroacetabular impingement may cause irritation and/or damage to the labrum or articular cartilage of the hip joint. Femoroacetabular impingement may result from a bony overgrowth around the acetabular rim 114, which results in a pincer deformity 116 (left view). In other cases, femoroacetabular impingement may result from a bony overgrowth from the femur 102, and particularly from the femoral neck 108 proximate to the femoral head 110, which results in a cam deformity 118 (middle view). In yet still other cases, there may be both a pincer deformity 116 and a cam deformity 118 (right view).

The bony overgrowth from the femoral neck 108 may extend from the femoral neck 108 in any radial direction relative to a longitudinal central axis of the femoral neck 108, though in most cases the bony overgrowth is more prominent on the superior and anterior surfaces. The bony overgrowth from the acetabular rim 114 may extend from the acetabular rim 114 at any location around the socket 112, though in most cases the bony overgrowth is more prominent on the superior surfaces and extending toward the femoral neck 108. The point is, pincer deformities and cam deformities may be disposed at any location around the femoral neck 108 and/or acetabular rim 114. A fluoroscopic image only shows a silhouette of the hip joint, and thus in the related art, during the surgery many surgeons generate the fluoroscopic images from a plurality of angles in an attempt to gauge the amount of bone that remains to be removed to correct the impingement.

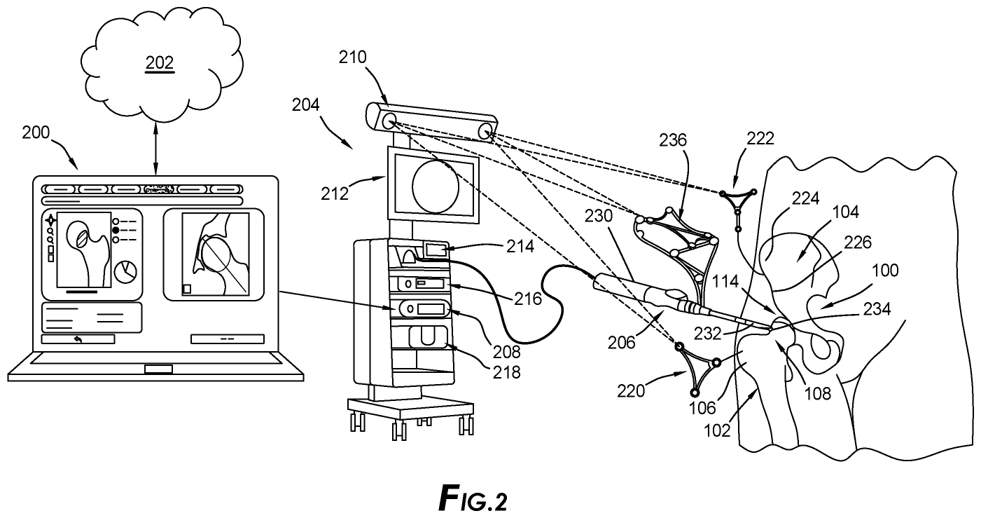

FIG. 2 shows a system in accordance with at least some embodiments. In particular, FIG. 2 shows a planning computer 200, a cloud computer 202, a device cart 204, an example patient showing the patient's hip joint 100, and a resection device 206 in operational relationship to the hip joint 100. Each will be addressed in turn.

In example systems, the planning computer 200 and cloud computer 202 may be used during the preoperative planning to perform a variety of preoperative tasks. In some examples, the software for the preoperative planning aspects are executed in the cloud computer 202 and accessed by way of the planning computer 200, which may be any suitable computer such as desktop, laptop, tablet computer, or smart phone device. For example, the planning computer 200 and/or the cloud computer 202 may receive a plurality of images of the hip joint 100. The images may be X-ray images, computed tomography (CT) images, ultrasound images, magnetic resonance imaging (MRI) images, or combinations. In example systems, the planning computer 200 and/or the cloud computer 202 may create from the images a three-dimensional model of the exterior surface of the femur 102, a three-dimensional model of the acetabulum 104, or both. As for the femur 102, the three-dimensional model may comprise the upper or superior portions of the femur 102. As for the acetabulum 104, the three-dimensional model may comprise only relevant portions of the acetabulum 104 (e.g., just portions of the hip joint 100 at issue).

Using the planning computer 200 and/or the cloud computer 202, in example systems the surgeon may create a resection plan for the upcoming surgery, or modify a resection plan automatically generated, but in either case the resection plan resulting in a planned resection volume associated with the hip joint 100. The planned resection volume represents a volume of bone to be removed from the femoral neck 108, a volume of bone to be removed from the acetabular rim 114, or both. The planned resection volume may take any suitable form. For example, the planned resection volume may be represented by two three-dimensional models of the hip joint 100: the first three-dimensional model being a preoperative surface model including the bony overgrowth(s); and the second three-dimensional model being the planned postoperative surface model with the bony overgrowth(s) removed. In other cases, the planned resection volume may be a three-dimensional volume directly representing the bone to be removed from the starting point of the preoperative surface model of the target member of the hip joint 100. In yet still further cases, the planned resection volume may be a three-dimensional volume representing the bone to be removed relative to the planned postoperative surface model of the target member of the hip joint 100. Regardless of the precise nature of the three-dimensional surface model and the planned resection volume, once preoperatively established the three-dimensional surface model and the planned resection volume may be transferred to a procedure controller (discussed more below) for use during the intraoperative portion of the example methods.

Still referring to FIG. 2, the example system further comprises the device cart 204. The device cart 204 may be used in the surgical setting during the intraoperative portion of the example methods. The device cart 204 may comprise a procedure controller 208, a stereoscopic camera 210 coupled to the procedure controller 208, a display device 212 coupled to the procedure controller 208, and a resection controller 214 communicatively coupled to the procedure controller 208. Other devices and controllers may be present as part of the device cart 204, such as an endoscopic light source and video controller 216 (hereafter just video controller 216), and peristaltic pump system 218 which may be used to control inflow and outflow within the hip joint 100. The example device cart 204 shows only a single display device 212 used by the procedure controller 208; however, in practice a second display device may be present to shows video images created by an endoscope or arthroscope (not shown). The second display device may take any suitable form, such as a duplicate display, or a head-mounted display implementing an Augmented Reality (AR) or Virtual Reality (VR) system. In yet still other cases, the display device 212 may be shared by the procedure controller 208 and the video controller 216 associated with the arthroscope.

The stereoscopic camera 210 may take any suitable form. In some cases, the stereoscopic camera 210 is designed and constructed to receive light within the infrared (IR) band of frequencies, but in other cases the stereoscopic camera 210 may be operable with light in the visible range, or both. Regardless, in being stereoscopic, the stereoscopic camera 210 may be used by the procedure controller 208 to monitor location of various devices and structures in the surgical room of a three-dimensional coordinate space. That is, example systems either operate based on ambient light within the surgical room, or shine light toward the surgical procedure (e.g., IR frequencies). The light of interest is reflected by reflectors of fiducial arrays, and based on the reflected light the procedure controller 208 may determine the location of the fiducial arrays (and their attached devices/structures). In yet still other examples, the fiducials of fiducial arrays may actively emit light at relevant frequencies for capture by the stereoscopic camera 210. For example, prior to the resection the surgeon may mechanically and rigidly couple a femur fiducial array 220 to the femur 102, such as by coupling the femur fiducial array 220 to the greater trochanter 106 of the femur 102. Once the femur fiducial array 220 is attached, and the femur 102 is correlated or registered to a three-dimensional model of the femur 102, the procedure controller 208 may monitor location of the femur fiducial array 220, and thus the femur 102, within the three-dimensional coordinate space of the surgical room.

As another example of monitoring location of various devices and structures in a three-dimensional coordinate space, prior to resection the surgeon may mechanically and rigidly couple an acetabular fiducial array 222 to the acetabulum 104. The acetabular fiducial array 222 may be coupled at any suitable location, such as the superior iliac spine 224 or the inferior iliac spine 226, or both. Once the acetabular fiducial array 222 is coupled to the acetabulum 104, and the acetabulum 104 is correlated or registered to a three-dimensional model of the acetabulum 104, the procedure controller 208 may monitor the location of the acetabular fiducial array 222, and thus the acetabulum 104, within a three-dimensional coordinate space. While FIG. 2 shows a system in which the procedure controller 208 monitors location of the both the femur 102 and the acetabulum 104, in some case only one member of the hip joint 100 may be monitored, such as monitoring just the femur 102 when only a cam-deformity is to be resected, or monitoring just the acetabulum 104 when only a pincer-deformity is to be resected.

Still referring to FIG. 2, the procedure controller 208 may monitor, using the stereoscopic camera 210, location of the resection device 206 operatively coupled to the resection controller 214. More particularly, in example systems the procedure controller 208 may monitor location of a distal end of the of the resection device 206 to track an amount of bone removed during resection. The example resection device 206 comprises a motor drive unit (MDU) or handpiece 230, an elongate outer tube 232 coupled to and extending from the handpiece 230, and a cutter 234 on a distal end of the elongate outer tube 232. In one example, the resection device 228 is a mechanical resection device in which the cutter 234 is a burr, but any suitable mechanical resection device may be used. In order to track the distal end (e.g., the cutter 234) of the resection device 228, an optical tracking array 236 is coupled to the resection device 228 in optical view of the stereoscopic camera 210. By monitoring location of the cutter 234 in the three-dimensional coordinate space, the procedure controller 208 may perform several advantageous tasks. For example, the procedure controller 208 may track an amount of bone resected, and the procedure controller 208 may control rate of resection of the resection device 206. Each will be addressed in turn.

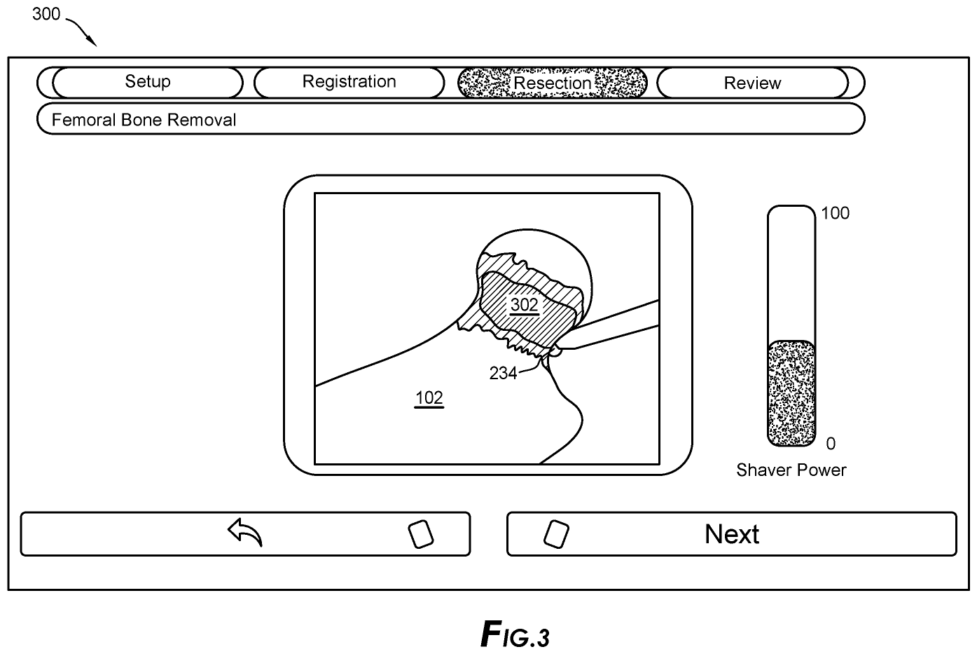

FIG. 3 shows an example user interface, in accordance with at least some embodiments. In particular, the example user interface 300 may be displayed on the display device 212 (FIG. 2) of the device cart 204 (also FIG. 2) during an intraoperative procedure to treat femoroacetabular impingement. The example user interface 300 is shown in the context of bone resection from a femur to treat a cam deformity, but the display techniques and related features are equally applicable to bone resection from the acetabulum to treat pincer deformities. FIG. 3 thus depicts a portion of the femur 102. The portion of the femur 102 depicted may be rendered from the three-dimensional model created during the preoperative planning (e.g., by the planning computer 200 and/or the cloud computer 202). FIG. 3 further shows a planned resection volume 302 in relation to the femur 102. In particular, the planned resection volume 302 is rendered on the depiction of the femur 102 to give a visual indication of the location and amount of bone to be removed. In FIG. 2, the planned resection volume 302 shows two example regions of varying resection by two patterns of crosshatching, but in practice one or more regions may be present in the planned resection volume. The example higher-density crosshatching shows greater volume to be removed, and the lower-density crosshatching shows lesser volume to be removed. Stated equivalently, in the region of higher-density crosshatching, the depth of bone to be removed is greater than the depth of bone to be removed in the region of lower-density crosshatching. In practice, the regions of the planned resection volume may be shown in a color-coded format. For example, regions with more bone to be removed may be shown in the “hotter” colors (e.g., reds and pinks), and regions with less bone to be removed may be shown in “cooler” colors (e.g., blues and greens).

FIG. 3 further shows a depiction of the cutter 234 of the resection device 206 (FIG. 2) in relation to the example femur 102 and the planned resection volume 302. In particular, in accordance with example systems the procedure controller 208 (FIG. 2) is designed and constructed to monitor location of the resection device 206 within the three-dimensional coordinate space by tracking the optical tracking array 236 (FIG. 2). Knowing the location of the cutter 234 in relation to each face of the optical tracking array 236 (e.g., by a registration process), the procedure controller 208 may calculate the location of the cutter 234 relative to the location of the three-dimensional model of the member of hip joint (here the femur 102) and/or the planned resection volume 302. The example procedure controller 208 may then render a depiction of a portion of the resection device 206 (e.g., the cutter 234 and elongate shaft as shown) in relation the example femur 102 and planned resection volume 302.

Further still, in example systems the procedure controller 208 (FIG. 2) may track an amount of bone removed or resected by the cutter 234 of the resection device 206 (FIG. 2). That is, because the example procedure controller 208 monitors location of the cutter 234 relative to the planned resection volume 302, when the cutter 234 abuts the planned resection volume 302 and the resection device 206 is operational, the procedure controller 208 is designed and constructed to assume that a predetermined amount of bone is removed from the location where the cutter 234 abuts the planned resection volume 302. In some cases, the procedure controller 208 is provided, prior to the intraoperative procedure, a value indicative of a rate of bone resecting as a function of contact time. In yet still other cases, the procedure controller 208 is provided, prior to the intraoperative procedure, a value indicative of a rate of bone resecting as a function of rotational speed of the cutter 234 and contact time. In other embodiments, the procedure controller 208 assumes any collision or overlap between the location of the cutter 234 in the rendering and the voxels of the bone model results in bone removal. In yet another approach, the procedure controller 208 performs noted overlap check but the bone model updates only occur when the cutter 238 is rotating. Regardless of the precise nature of the predetermined resection rate, as the cutter 234 interacts with the planned resection volume 302, the procedure controller 208 tracks an amount of bone removed using the predetermined resection rate.

In accordance with example systems, the procedure controller 208 (FIG. 2) may be designed and constructed to update the user interface 300 as bone is resected. At the high level, as bone is removed the user interface 300 may update the visual indication of the planned resection volume 302 to show the remaining bone to be removed. Considered again in terms of the planned resection volume 302 depicted with color, as bone is removed the procedure controller 208 may change the color within the visual depiction of the planned resection volume 302 by making the colors “cooler.” If an area is initially a “hotter” color indicating a greater volume of bone to be removed, then as the bone is removed the remaining bone may be shown in progressively “cooler” colors (e.g., pink becomes blue, blue becomes green). When all the bone from a planned resection volume has been removed, the procedure controller 208 may show exposed bone as white on the screen, matching the remaining portions of the bone outside the planned resection volume. It too much bone is removed, the exposed bone may appear in a different, alert, or warning color (e.g., red). In yet still other cases, additional auditory and visual changes may be implemented when the cutter 234 abuts an area where too much bone has been removed. Stated in terms of the planned resection volume, initially the procedure controller 208 shows a depiction of the planned resection volume 302. As bone is removed, the planned resection volume is updated to effectively become a remaining resection volume to be removed, and it is the remaining resection volume to be removed that is depicted on the display device 212 (FIG. 2) as shown in FIG. 3. The specification now turns to controlling a rate of resection based on the physical relationship between the cutter 234 and the planned resection volume 302.

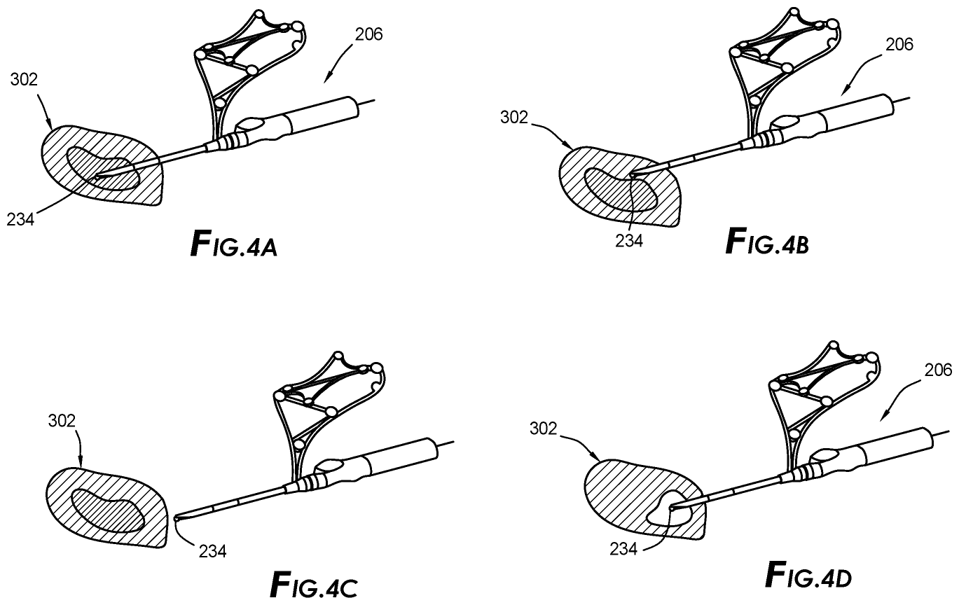

FIGS. 4A, 4B, 4C, and 4D show a distal end of a resection device in relation to a planned resection volume in accordance with at least some embodiments. Considering initially FIG. 4A, FIG. 4A shows an example planned resection volume 302 having two regions of varying resection volume, the regions of varying resection volume shown by two patterns of crosshatching. As before, the example higher-density crosshatching shows areas of greater volume to be removed, and the lower-density crosshatching shows areas of lesser volume to be removed. FIG. 4A further shows a cutter 234 of the resection device 206 abutting the planned resection volume 302. By monitoring location of the cutter 234 in the three-dimensional coordinate space, the procedure controller 208 (FIG. 2) may also control rate of resection of the resection device 206 based on the location of the cutter 234 relative to the planned resection volume 302. In FIGS. 4A-4C, the planned resection volumes 302 are shown to be the same, indicating that resection has just begun. FIG. 4D, however, shows an intermediate state of the planned resection volume 302 in which some bone volume has been removed, including an area in the lower right of the planned resection volume in which all the bone planned to be removed has been removed (and thus no crosshatching is present).

In some example cases, controlling the rate of resection may comprise controlling the rotational speed of the cutter 234 based on location of the cutter 234 relative to the planned resection volume 302. Consider first movement of the cutter 234 relative to the planned resection volume 302 as shown by FIGS. 4A and 4C. In FIG. 4A, the cutter 234 abuts the planned resection volume 302, and in FIG. 4C the cutter 234 has moved outside the boundary of the planned resection volume 302, meaning the cutter 234 in FIG. 4C abuts bone that should not be removed. In accordance with at least some embodiment, the procedure controller 208 (FIG. 2) is designed and constructed to reduce the rotational speed of the cutter 234 to zero responsive to the cutter 234 abutting bone outside the planned resection volume 302. Stated otherwise, in example systems the procedure controller 208 monitors location of the cutter 234 relative to the planned resection volume 302, and the procedure controller 208 turns the resection device off (e.g., by communication with the resection controller 214 (FIG. 2)) when the cutter 234 abuts bone outside the planned resection volume 302.

Still considering example cases of controlling the rate of resection based on location of the cutter 234 relative to the planned resection volume 302, now consider movement of the cutter 234 relative to the planned resection volume 302 as shown by FIGS. 4A and 4D. In FIG. 4A, the cutter 234 abuts the planned resection volume 302 in areas where bone still needs to be removed, and in FIG. 4D the cutter 234 abuts an area of bone in which all the bone planned to be removed has been removed, meaning the cutter 234 now abuts bone that should not be removed. In accordance with at least some embodiment, the procedure controller 208 (FIG. 2) is designed and constructed to reduce the rotational speed the cutter 234 to zero responsive to the cutter 234 abutting bone that should not be removed. Stated otherwise, in example systems the procedure controller 208 monitors location of the cutter 234 relative to the planned resection volume 302, and the procedure controller 208 turns the resection device off (e.g., by communication with the resection controller 214 (FIG. 2)) when the cutter 234 abuts bone beneath the planned resection volume 302.

In addition to turning the resection device 206 off when the cutter 234 abuts bone that should not be removed (e.g., outside the planned resection volume 302 or beneath the planned resection volume 302), further example embodiments may provide haptic feedback to the surgeon and/or audible feedback to the surgeon to give an indication of the location of the cutter 234 in relation to the planned resection volume 302. Regarding haptic/audible feedback, consider movement of the cutter 234 relative to the planned resection volume 302 as shown by FIGS. 4A and 4B. In FIG. 4A, the cutter 234 abuts the planned resection volume 302 in an area where greater bone volume needs to be removed, and in FIG. 4B the cutter 234 abuts an area where lesser bone volume needs to be removed. In accordance with at least some embodiments, the procedure controller 208 (FIG. 2) is designed and constructed to change the rotational speed the cutter 234 responsive to the cutter 234 abutting areas of differing volumes of bone to be removed. For example, when the cutter 234 abuts a portion of the planned resection volume 302 having greater bone to be removed (e.g., FIG. 4A), the procedure controller 208 may command the resection controller 214 (FIG. 2) to drive the cutter 234 at a first rotational speed. However, as the cutter 234 moves relative to the planned resection volume 302 to abut a portion having lesser bone to be removed (FIG. 4B), the procedure controller 208 may command the resection controller to drive the cutter 234 at a second rotational speed slower than the first rotational speed. Oppositely, when the cutter 234 abuts a portion of the planned resection volume 302 having lesser bone to be removed (e.g., FIG. 4B), the procedure controller 208 may command the resection controller 214 (FIG. 2) to drive the cutter 234 at a first rotational speed, and as the cutter 234 moves relative to the planned resection volume 302 to abut a portion having greater bone to be removed (FIG. 4A), the procedure controller 208 may command the resection controller 214 to drive the cutter 234 at a second rotational speed faster than the first rotational speed. Such a speed control mechanism may thus provide haptic feedback to the surgeon in the form of vibration of the resection device 206 relative to the location of the cutter 234 within the planned resection volume 302. Similarly, and given that change of rotational speed may also produce changes in audible sound created by the resection device 206, such a speed control mechanism may thus provide audible feedback to the surgeon as to the location of the cutter 234 within the planned resection volume 302. In such examples the rate of resection may be higher when the cutter 234 abuts locations where more bone is to be removed.

In addition to or in place of the audible feedback based on the speed of the cutter 234, the procedure controller 208 may have a sound-producing device or speaker that produces audible sound as based on location of the cutter 234 in relation to the planned resection volume. In yet still further embodiments, the speed control aspects can be disabled, leaving rotational speed of the cutter 234 solely to the discretion of the surgeon (e.g., based the surgeon interacting with a foot pedal or a buttons on the handpiece). In such cases, the procedure controller 208 may nevertheless track location of the cutter 238 in relation to the planned resection volume and when the cutter 238 is outside or below the planned resection volume the procedure controller 208 may provide an audible and/or visual alarm, but leave the rotational speed of the cutter 238 unchanged.

The example haptic and/or audible feedback to the surgeon may also be used to inform the surgeon of proximity of the cutter 234 to the outer boundary of the planned resection volume 302. Consider movement of the cutter 234 relative to the planned resection volume 302 as shown by FIGS. 4A, 4B, and 4C. In FIG. 4A, the cutter 234 abuts the planned resection volume 302 near the center of the planned resection volume. In FIG. 4B, the cutter 234 has been moved from near the center closer to a boundary of the planned resection volume. And in FIG. 4C the cutter 234 has moved beyond the boundary of the planned resection volume 302. In accordance with at least some embodiments, the procedure controller 208 (FIG. 2) is designed and constructed to change the rotational speed the cutter 234 responsive to the movement of the cutter 234 toward the boundary of the planned resection volume 302. For example, when the cutter 234 is near the center of the planned resection volume 302, the procedure controller 208 may command the resection controller 214 (FIG. 2) to drive the cutter 234 at a rotational speed. However, as the cutter 234 moves closer to the boundary of the planned resection volume 302, the procedure controller 208 may command the resection controller to drive the cutter 234 at progressively slower rotational speeds as a function of how close the cutter 234 is to the boundary. In some cases, the procedure controller 208 may then reduce the speed of the cutter 234 to zero as the cutter 234 crosses the boundary of the planned resection volume. Oppositely, as the cutter 234 moves farther from the boundary of the planned resection volume 302, the procedure controller 208 may command the resection controller to drive the cutter 234 at progressively faster rotational speeds as a function of how far the cutter 234 is from the boundary. Such a speed control mechanism may thus provide haptic feedback to the surgeon, in the form of vibration of the resection device 206, as to the location of the cutter 234 within the planned resection volume 302. Similarly, and given that change of rotational speed may also produce changes in audible sound created by the resection device 206, such a speed control mechanism may thus provide audible “speakerless” feedback to the surgeon as to the location of the cutter 234 within the planned resection volume 302. A similar audible feedback may be implemented with the sound producing element or speaker. The specification now turns to generating simulate fluoroscopic images to aid the surgeon in determining whether sufficient bone has been removed.

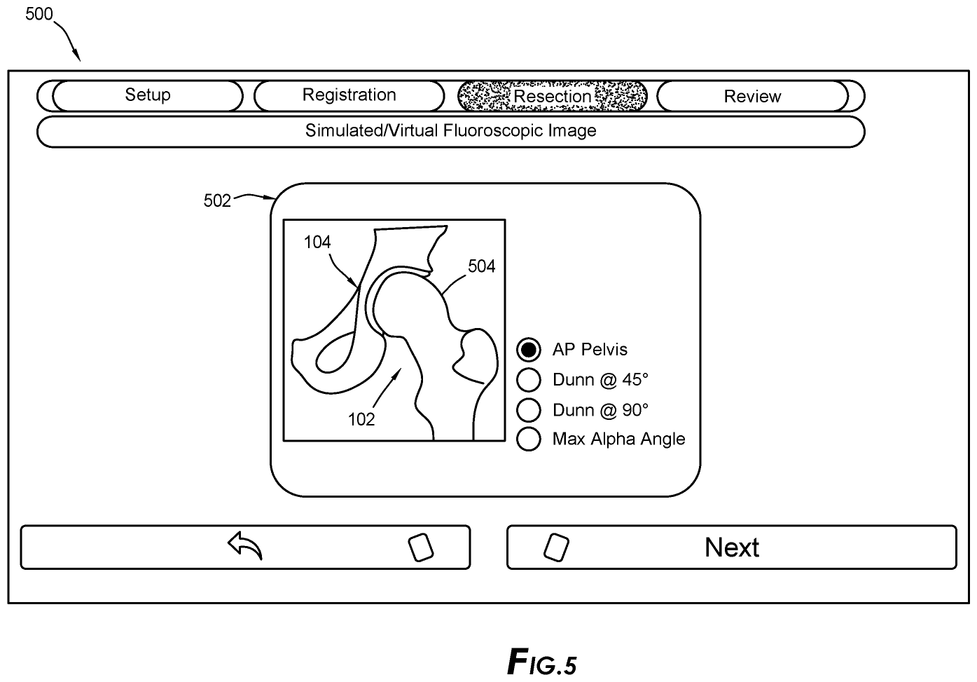

FIG. 5 shows an example user interface, in accordance with at least some embodiments. In particular, the user interface 500 may be displayed on the display device 212 (FIG. 2) of the device cart 204 (also FIG. 2) during an intraoperative procedure to treat femoroacetabular impingement. The example user interface 500 is shown in the context of bone resection from a femur to treat a cam deformity, but the display techniques and related features are equally applicable to bone resection from the acetabulum to treat pincer deformities. The user interface 500 of FIG. 5 shows a virtual or simulated fluoroscopic image 502 including a portion of the femur 102 having a cam deformity 504. In accordance with example cases, the simulated fluoroscopic image may be rendered from the three-dimensional model created during the preoperative planning (e.g., by the planning computer 200 and/or the cloud computer 202), and taking into account the amount of bone that has been removed.

As discussed above, in example embodiments the procedure controller 208 (FIG. 2) is designed and constructed to monitor location of the cutter 234 in relation to the planned resection volume, and to track an amount of bone resected. Based on the tracking of the amount of bone resected, the procedure controller 208 may be designed and constructed to generate the simulated fluoroscopic image 502 that shows the hip joint as the hip joint would look after the amount of bone has been removed. In accordance with example embodiments, the procedure controller 208 may generate the simulated fluoroscopic image from any of a variety of viewpoints and any of a variety of hip flexions, all to aid the surgeon in gauging whether sufficient bone has been removed to address the femoroacetabular impingement. In some cases, the surgeon may forgo actual fluoroscopic imaging and use the simulated fluoroscopic image 502 alone. In other cases, the surgeon may use the simulated fluoroscopic image 502 as an initial guide, and then verify with intraoperative fluoroscopic imaging.

In FIG. 5, a single fluoroscopic image is shown, and thus the simulated fluoroscopic image 502 of FIG. 5 may be equivalently stated to be a single X-ray image. However, in yet still further cases, the example procedure controller 208 (FIG. 2) may render and display a series of images, and thus may produce simulated fluoroscopic imaging, including showing movement of the femur 102 relative the acetabulum 140 to show potential impingement issues from any suitable vantage point.

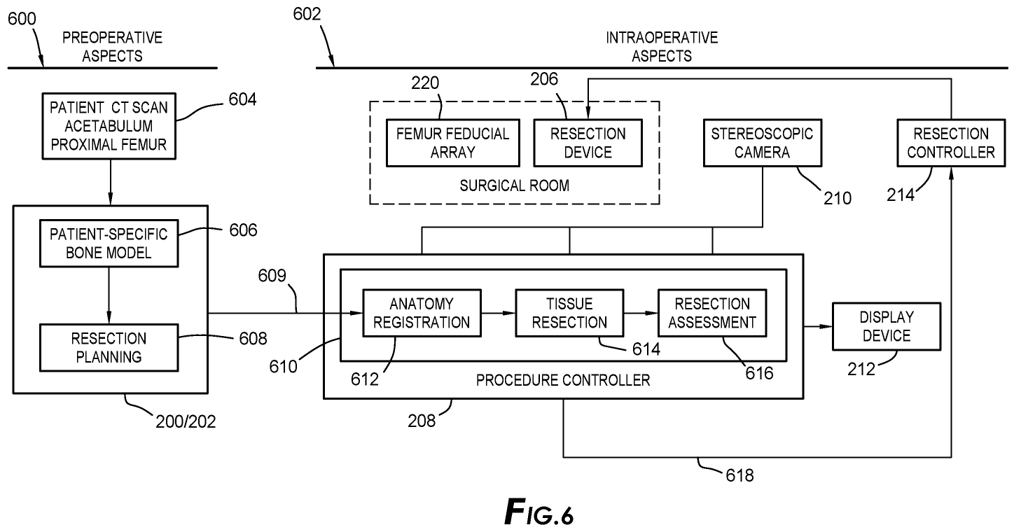

FIG. 6 shows a partial block diagram, and partial flow diagram, of a system for treating femoroacetabular impingement. In particular, FIG. 6 is conceptually organized into preoperative planning aspects 600 and intraoperative procedure aspects 602. From a system standpoint, FIG. 6 shows, in block diagram form, the planning computer 200 and/or cloud computer 202, the procedure controller 208, the display device 212, the stereoscopic camera 210, the resection controller 214, the resection device 206, and the femur fiducial array 220. In other cases, the fiducial array may be the acetabular fiducial array.

The planning computer 200 and/or the cloud computer 202 are provided a plurality of images during the preoperative planning aspects 600. In FIG. 6, being provided the plurality of images is shown by block 604, designating CT scan images, but any suitable image type or combination of image types may be provided to the planning computer 200 and/or the cloud computer 202. The example planning computer 200 and/or the cloud computer 202 may execute a bone-modelling software 606 and a resection planning software 608. The bone-modelling software 606 is designed and constructed to create the three-dimensional model of at least a portion of the hip joint based on a plurality of images (from block 604), with the three-dimensional model in any suitable form as discussed above. The resection planning software 608 is designed and constructed to create the planned resection volume. Prior to the intraoperative procedure aspects 602, the planning computer 200 and/or the cloud computer 202 may transfer three-dimensional model of the bone and the planned resection volume to the procedure controller 208, as shown by arrow 609. The transfer of the three-dimensional model of the bone and the planned resection volume may take any suitable form, such as by way of an Ethernet connection, a directly coupled serial communication protocol, a wireless point-to-point connection (e.g., Bluetooth), or transferring using a memory device (e.g., a Universal Serial Bus (USB) solid-state drive).

Still referring to FIG. 6, and specifically now the intraoperative procedure aspects 602. The example procedure controller 208 is operatively coupled to the display device 212 to display any of the example user interfaces discussed above. Additionally, the procedure controller 208 is operatively coupled to the stereoscopic camera 210 to receive stereoscopic images of the procedure field, including stereoscopic images of the optical tracking array 236 (FIG. 2) of the resection device 206 and stereoscopic images of the fiducial arrays coupled to bone, which in the example case of FIG. 6 is the femur fiducial array 220. Further still, the example procedure controller 208 is operatively coupled to the resection controller 214 in any suitable form (e.g., Universal Serial Bus (USB), controller area network (CAN) bus). By way of the connection to the resection controller 214, the procedure controller 208 may control rotational speed of the cutter of the resection device 206 as a function of location of the cutter relative to the planned resection volume. In some cases, the procedure controller 208 may control rotational speed of the cutter of the resection device 206 as a function of location of the cutter relative to the planned resection volume and the speed requested by the surgeon (such as by interaction with a foot pedal). The slower of the rotational speeds indicated by the location of the cutter and the foot pedal may the rotational speed actually implemented.

Operatively, the procedure controller 208 executes resection control software 610. The resection control software 610 is conceptually, though not necessarily physically, divided into three example components: anatomy registration software 612; tissue resection software 614; and resection assessment software 616. The anatomy registration software 612 is used during the registration process. Consider, as an example, an intraoperative procedure to remove a cam deformity from the femoral neck. During the registration process, the procedure controller 208 correlates the three-dimensional model of the bone provided by the planning computer 200 and/or cloud computer 202 to the actual femur by tracking femur fiducial array 220 as the surgeon touches various points on the femur with a probe and corresponding probe fiducial array (the probe and corresponding probe fiducial array not shown so as not to unduly complicate the figure). Once the registration process is complete, the example intraoperative procedure aspects 602 may proceed to bone resection.

Still referring to FIG. 6, during bone resection the tissue resection software 614 performs a variety of tasks. For example, the tissue resection software 617 may monitor location of a member of an acetabulofemoral joint (e.g., the femur using the femur fiducial array 220) in a three-dimensional coordinate space, the monitoring using the stereoscopic camera 210. Similarly, the tissue resection software 617 may monitor location of the cutter of the resection device 206 in the three-dimensional coordinate space, the monitoring using the stereoscopic camera 210 and the optical tracking array 236 (FIG. 2) associated with the resection device 206. Based on the location of the member of the hip joint and the location of the cutter of the resection device 206, the example tissue resection software 614 may track an amount of bone resected from the member of the hip joint. Further, the tissue resection software 614 may control the rate of resection of the resection device 206 based on the location of the cutter of the resection device 306 relative to the planned resection volume. Controlling the rate of resection may take any of the forms discussed above, including reducing the rate of resection to zero (e.g., turning the resection device off) when the cutter of the resection device 206 resides outside or below the planned resection volume. Controlling the rate of resection may be implemented by the tissue resection software 614 of the procedure controller 208 communicating with the resection controller 214 as shown by line 618.

Though FIG. 6 shows the resection assessment software 616 executing after the tissue resection software 614 for convenience of the figure, in practice the resection assessment software 616 may be executed simultaneously or in parallel with the tissue resection software 614. The resection assessment software 616 may be designed and constructed to create the various user interfaces discussed above, such as user interface 300 showing the three-dimensional bone model, the planned resection model (including remaining bone to be resected), and a visual indication of the location of the cutter relative to the planned resection volume. Additionally or in place of the user interface 300 of FIG. 3, the resection assessment software 616 may create the user interface 500 showing either a single virtual fluoroscopic image, or a series of virtual fluoroscopic images including movement of the hip joint, with the images showing the underlying bones as the image(s) would look taking into account the amount of bone that has already been resected.

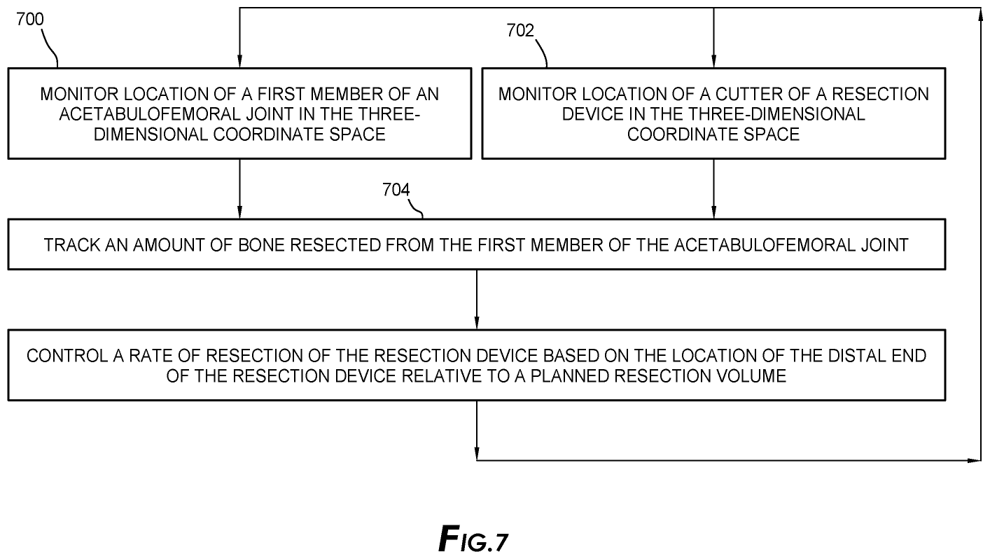

FIG. 7 shows a method of treating femoroacetabular impingement in accordance with at least some embodiments. Some or all the method may be implemented by a processor executing software. In particular the example method comprises monitoring location of a first member of an acetabulofemoral joint in the three-dimensional coordinate space (block 700). Simultaneously with monitoring location of the joint, the example method comprises monitoring location of a cutter of a resection device in the three-dimensional coordinate space (block 702). The example method then comprises tracking an amount of bone resected from the first member of the acetabulofemoral joint (block 704). From there, the example method comprises controlling a rate of resection of the resection device based on the location of the distal end of the resection device relative to a planned resection volume (block 708). Thereafter, the method repeats during the intraoperative procedure aspects of treating the femoroacetabular impingement.

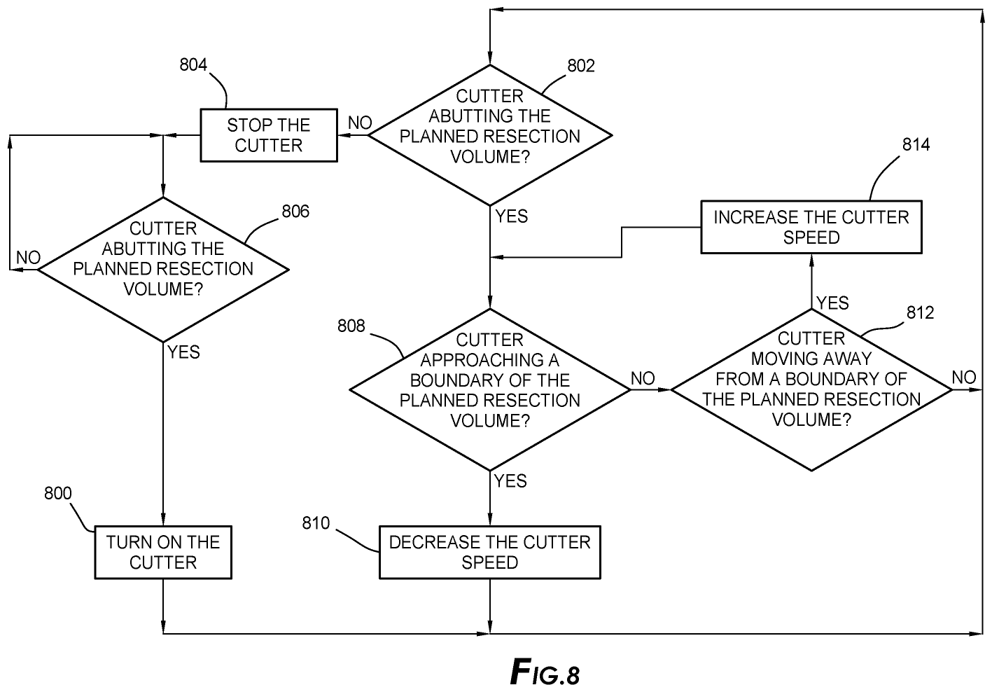

FIG. 8 shows a method of controlling rate of resection of the resection device in accordance with at least some embodiments. Some or all the method may be implemented by a processor executing software. In particular, FIG. 8 assumes the cutter is initially placed against the planned resection volume, and that the surgeon has turned the cutter on (e.g., instigated rotation of the cutter element), and thus conceptually with the method may begin at block 800. From turning on the cutter (again block 800), the example method comprises determining whether the cutter is abutting the planned resection volume (block 802). If at any point the cutter abuts bone outside the perimeter of the planned resection volume or bone beneath the planned resection volume bone (again block 802), the example method proceeds to stopping the cutter (block 804). With the cutter stopped, the example method continues to the track the location of the cutter in relation to the planned resection volume to determine when the cutter again abuts bone within the planned resection volume (block 806), and when the cutter again abuts bone with the planned resection volume the cutter is again turned on (again block 800).

Still referring to FIG. 8, and this time starting at the determination of whether the cutter is abutting the planned resection volume (of block 802). Assuming the cutter is still abutting the planned resection volume (the “YES” path out of decision block 802), the next example determination is whether the cutter is approaching a boundary of the planned resection volume (block 808). The boundary of the planned resection volume may include not only the outer perimeter of the planned resection volume, but may also including the lower boundary of the planned resection volume beneath which further bone should not be removed. The determination as to approaching the boundary may take any suitable form, such as a determination that the cutter is within a predetermined distance (e.g., 2 mm to 8 mm inclusive) of an outer perimeter of the planned resection volume, or determining the cutter is within a predetermined distance from an inner perimeter if enough bone has been removed (e.g., FIG. 4D). Regardless of the precise nature of the boundary at issue or the distance to the boundary, when a boundary is approached (the “YES” path out of the decision block 808), the example method slows the cutter speed (block 810) to reduce the rate of resection, provide haptic/audible feedback to the surgeon that a boundary is being approached, or both. From there, the example method returns to the determination as to whether cutter is abutting the planned resection volume (again block 802). Assuming for purposes of explanation that the cutter still abuts the planned resection volume (the “YES” path out of decision block 802), once again the example method makes the determination of whether the cutter is approaching the boundary of the planned resection volume (again block 808). If the cutter is still approaching the boundary, yet another speed adjustment (block 810) may be made by the example method.

Still referring to FIG. 8, now consider that the cutter is not approaching a boundary of the planned resection volume (the “NO” path out of decision block 808). The example method may make a determination as to whether the cutter is moving away from a boundary of the planned resection volume (block 812). The determination as to whether the cutter is moving away from a boundary may take any suitable form. For example, the cutter moving a predetermined distance (e.g., 2 mm to 8 mm inclusive) away from any boundary of the planned resection volume may be an indication the cutter is moving away from a boundary. In other cases, when the average distance of the cutter to the closest boundary of the planned resection volume is increasing, such may be an indication that the cutter is moving away from a boundary. Regardless of the precise nature of the boundary at issue or the distance to the boundary, if the cutter is moving away from a boundary (the “YES” path out of decision block 812), then the example method may increase the speed of the cutter (block 814) and then enter again the decision block 808. In summary then, as the cutter moves generally away from a boundary of the planned resection volume, cutter speed may be increased to a predetermined speed, and as the cutter approaches a boundary, the cutter speed may be decreased.

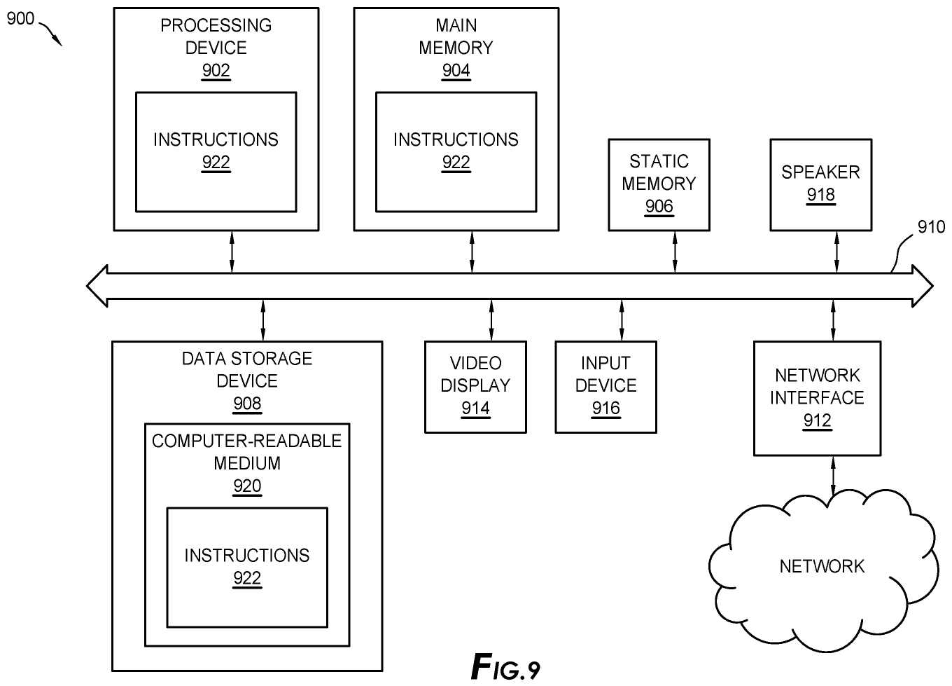

FIG. 9 shows an example computer system 900. In one example, computer system 900 may correspond to the planning computer 200, the cloud computer 202, or the procedure controller 208. The computer system may be connected (e.g., networked) to other computer systems in a local-area network (LAN), an intranet, an extranet, or the Internet. The computer system 900 may be a personal computer (PC), a tablet computer or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 900 includes a processing device 902, a main memory 904 (e.g., read-only memory (ROM), flash memory, dynamic random access memory (DRAM) such as synchronous DRAM (SDRAM)), a static memory 906 (e.g., flash memory, static random access memory (SRAM)), and a data storage device 908, which communicate with each other via a bus 910.

Processing device 902 represents one or more general-purpose processing devices such as a microprocessor, central processing unit, or the like. More particularly, the processing device 902 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 902 may also be one or more special-purpose processing devices such as an application specific integrated circuit (ASIC), a field programmable gate array (FPGA), a digital signal processor (DSP), network processor, or the like. The processing device 902 is configured to execute instructions for performing any of the operations and steps discussed herein. Once programmed with specific instructions, the processing device 902, and thus the entire computer system 900, becomes a special-purpose device.

The computer system 900 may further include a network interface device 912. The computer system 900 also may include a video display 914 (e.g., a display device 212, or the display device associated with the planning computer 200 of FIG. 2), one or more input devices 916 (e.g., a keyboard and/or a mouse), and one or more speakers 918. In one illustrative example, the video display 914 and the input device(s) 916 may be combined into a single component or device (e.g., an LCD touch screen).

The data storage device 908 may include a computer-readable storage medium 920 on which the instructions 922 (e.g., implementing any methods and any functions performed by any device and/or component depicted described herein) embodying any one or more of the methodologies or functions described herein is stored. The instructions 922 may also reside, completely or at least partially, within the main memory 904 and/or within the processing device 902 during execution thereof by the computer system 900. As such, the main memory 904 and the processing device 902 also constitute computer-readable media. The instructions 922 may further be transmitted or received over a network via the network interface device 912.

While the computer-readable storage medium 920 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

1-10. (canceled)

11. A method of treating femoroacetabular impingement, the method comprising:
monitoring, by a procedure controller, location of a first member of an acetabulofemoral joint in a three-dimensional coordinate space;
tracking, by the procedure controller, an amount of bone resected by tracking a distal end of a resection device in the three-dimensional coordinate space; and
generating, by the procedure controller, a simulated fluoroscopic image that shows the first member of the acetabulofemoral joint after the amount of bone has been removed; and
displaying, on a display device, the simulated fluoroscopic image.

12. The method of claim 11 further comprising:
creating a three-dimensional model of at least a portion of the first member of the acetabulofemoral joint based on a plurality of images, the creating the three-dimensional model prior to resecting bone; and
providing the three-dimensional model to the procedure controller;
wherein generating the simulated fluoroscopic image further comprises creating the simulated fluoroscopic image based on the three-dimensional model and the amount of bone resected.

13. The method of claim 12 wherein the plurality of images are selected from a group comprising: x-ray images; computed tomography images; and magnetic resonance imaging images.

14. The method of claim 11 wherein generating the simulated fluoroscopic image further comprises generating a plurality of simulated fluoroscopic images, each image at a different angle relative to the acetabulofemoral joint.

15. The method of claim 11 further comprising controlling a rate of resection of the resection device based on the location of the distal end of the resection device relative to a planned resection volume associated with the first member of the acetabulofemoral joint.

16. The method of claim 15 wherein controlling the rate of resection further comprises decreasing a rotational speed of a cutter of the resection device when the distal end of the resection device resides on a portion of the planned resection volume having less than a predetermined amount of bone to be removed.

17. The method of claim 15 wherein controlling the rate of resection further comprises increasing a rotational speed of a cutter of the resection device when the distal end of the resection device resides on a portion of the planned resection volume having more than a predetermined amount of bone to be removed.

18. The method of claim 15 wherein controlling the rate of resection further comprises controlling a rotational speed of a cutter of the resection device based on location of the distal end of the resection device in relation to remaining bone to be removed in the planned resection volume.

19. The method of claim 18 wherein controlling the rate of resection further comprises changing a rotational speed of the cutter of the resection device to zero responsive to the distal end of the resection device abutting bone outside the planned resection volume.

20. The method of claim 18 wherein controlling the rate of resection further comprises changing a rotational speed of the cutter of the resection device to zero responsive to the distal end of the resection device abutting bone in an area beneath the planned resection volume.

21. A system for treating femoroacetabular impingement, the system comprising:
a procedure controller;
a stereoscopic camera coupled to the procedure controller;
a display device coupled to the procedure controller;
a resection controller communicatively coupled to the procedure controller;
a resection device operatively coupled to the resection controller, the resection device comprising a handpiece, an elongate outer tube coupled to and extending from the handpiece, and a cutter disposed on a distal end of the elongate outer tube; and
an optical tracking array coupled to the resection device and in optical view of the stereoscopic camera;
wherein the procedure controller is configured to:
monitor location of a first member of an acetabulofemoral joint in a three-dimensional coordinate space;
track an amount of bone resected from the first member of the acetabulofemoral joint by tracking a distal end of the resection device in the three-dimensional coordinate space; and
control a rate of resection of the resection device based on a location of the distal end of the resection device relative to a planned resection volume associated the first member of the acetabulofemoral joint.

22. The system of claim 21 wherein the procedure controller monitors the location of the first member of the acetabulofemoral joint, the procedure controller may be further configured to monitor at least one selected from a group comprising: a femur; and an acetabulum.

23. The system of claim 21 wherein the procedure controller controls the rate of resection, the procedure controller may be further configured to decrease a rotational speed of the cutter of the resection device when the distal end of the resection device resides on a portion of the planned resection volume having less than a predetermined amount of bone to be removed.

24. The system of claim 21 wherein the procedure controller controls the rate of resection, the procedure controller may be further configured to increase a rotational speed of a cutter of the resection device when the distal end of the resection device resides on a portion of the planned resection volume have more than a predetermined amount of bone to be removed.

25. The system of claim 21 wherein the procedure controller controls the rate of resection, the procedure controller may be further configured to control a rotational speed of the cutter of the resection device based on location of the distal end of the resection device in relation to remaining bone to be removed in the planned resection volume.

26. The system of claim 25 wherein when the procedure controller controls the rate of resection, the procedure controller may be further configured to control a rotational speed of the cutter of the resection device to zero responsive to the distal end of the resection device abutting bone outside the planned resection volume.

27. The system of claim 25 wherein when the procedure controller controls the rate of resection, the procedure controller may be further configured to control a rotational speed of the cutter of the resection device to zero responsive to the distal end of the resection device abutting bone outside the planned resection volume.

28. The system of claim 21 wherein the procedure controller is further configured to, prior to controlling the rate of resection, receive a three-dimensional model of at least a portion of the first member of the acetabulofemoral joint in three-dimensions based on a plurality of images; and receive a planned resection volume based on the three-dimensional model.

29. A system for treating femoroacetabular impingement, the system comprising:
a procedure controller;
a stereoscopic camera coupled to the procedure controller;
a display device coupled to the procedure controller;
a resection controller communicatively coupled to the procedure controller;
a resection device operatively coupled to the resection controller, the resection device comprising a handpiece, an elongate outer tube coupled to and extending from the handpiece, and a cutter disposed on a distal end of the elongate outer tube; and
an optical tracking array coupled to the resection device and in optical view of the stereoscopic camera;
wherein the procedure controller is configured to:
monitor location of a first member of an acetabulofemoral joint in a three-dimensional coordinate space;
track an amount of bone resected by tracking a distal end of the resection device in the three-dimensional coordinate space;
generate a simulated fluoroscopic image that shows the first member of the acetabulofemoral joint without the amount of bone resected; and
display, on the display device, the simulated fluoroscopic image.

30. The system of claim 29 wherein when the procedure controller generates the simulated fluoroscopic image, the procedure controller is further configured to generate a plurality of simulated fluoroscopic images, each image at a different angle relative to the acetabulofemoral joint.

31. The system of claim 29 wherein the procedure controller is further configured to control a rate of resection of the resection device based on a location of the distal end of the resection device relative to a planned resection volume associated with the first member of the acetabulofemoral joint.

32. The system of claim 31 wherein when the procedure controller controls the rate of resection, the procedure controller is further configured to decrease a rotational speed of the cutter of the resection device when the distal end of the resection device resides on a portion of the planned resection volume having less than a predetermined amount of bone to be removed.

33. The system of claim 31 wherein when the procedure controller controls the rate of resection, the procedure controller is further configured to increase a rotational speed of the cutter of the resection device as the distal end of the resection device resides on a portion of the planned resection volume having more than a predetermined amount of bone to be removed.

34. The system of claim 31 wherein when the procedure controller controls the rate of resection, the procedure controller is further configured to control a rotational speed of the cutter of the resection device based on location of the distal end of the resection device in relation to remaining bone to be removed in the planned resection volume.

35. The system of claim 34 wherein when the procedure controller controls the rate of resection, the procedure controller is further configured to change a rotational speed of the cutter of the resection device to zero responsive to the distal end of the resection device abutting bone outside the planned resection volume.

36. The system of claim 34 wherein when the procedure controller controls the rate of resection, the procedure controller is further configured to change a rotational speed of the cutter of the resection device to zero responsive to the distal end of the resection device abutting bone in an area beneath the planned resection volume.
